(19) United States
(12) Patent Application Publication
(10) Pub. No.: US 2005/0130237 A1
(43) Pub. Date: Jun. 16, 2005

(54) High throughput screening method for compounds with non-, pro-, or anti-apoptotic or proliferative or necrotic activity

(76) Inventors: Alessandro Strebel, Oberwil (CH); Felix Bachmann, Basel (CH); Thomas Harr, Allschwil (CH)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/502,477

(22) PCT Filed: Feb. 4, 2002
(86) PCT No.: PCT/CH02/00065

(51) Int. Cl. G01N 33/574

(52) U.S. Cl. 435/7.23

(57) ABSTRACT

The present ivention relates to a high through put screening method for assaying the non-, pro-, or anti-apoptonic or proliferative or necrotic activity of test compounds in cells, using vectors coding for a specific marker protein, cell lines and spheroids transfected with said vectors.

### FIELD OF THE INVENTION

The present invention relates to an improved and thus industrially applicable high through put screening method for assaying the non-, pro-, or anti-apoptotic or proliferative or necrotic activity of test compounds in cells, using vectors coding for a specific marker protein, primary cells, cell lines and spheroids transfected with said vectors. The herein described method allows for a clear and unambiguous assessment of non-, pro-, or anti-apoptotic or proliferative or necrotic activity of said test compounds in said cells.

### BACKGROUND OF THE INVENTION

Apoptosis plays an essential role in development, i.e. embryogenesis and normal cell turnover, but also in diseases such as cancer, autoimmune diseases, neurodegenerative and cardiovascular diseases and viral infections including AIDS. Unlike necrosis, apoptosis is an active, gene-directed self-destruction process of the cell and is associated with characteristic morphological and biochemical changes^{1, 2}. Nuclear and cytoplasmic condensation and fragmentation of the dying cell into membrane-bound apoptotic bodies are typical characteristics of apoptosis. Another feature of apoptotic cell death is the chromosomal DNA degradation into oligonucleosomal fragments after the activation of specific nucleases^{3, 4}. Apoptosis can be extrinsically imposed on cells through the interaction of the so-called death receptors with their corresponding ligands, such as Fas (also termed CD95 or Apo-1) with Fas-Ligand (FasL), TRAIL receptor (TRAIL-R) with TRAIL²⁶, tumor necrosis factor (TNF)-R1 or 2 with TNF, or intrinsically, by the activation of some members of the BCL-2 family. Other forms among a variety of apoptosis mediators include the Perforin/Granzyme system, cytokine deprivation (eg. IL-3 deprivation), irradiation (e.g. UV irradiation) and drug-induced apoptosis. Among the several different apoptotic pathways, it can be differentiated between caspase-dependent and caspase-independent apoptotic processes, which all end-up in the apoptotic death of the cell.

In contrast to apoptosis, necrosis is a non-physiological death of cells due to chemical or physical injury of the cell membrane. Morphological criteria include cell swelling and cell lysis, lysosomal leakage and loss of the cell membrane integrity.

During the last decade, it has become clear that apoptosis plays a keyrole in several diseases. Apoptosis is increased in AIDS, certain neurodegenerative and cardiovascular diseases but decreased in cancer and certain autoimmune proliferative diseases. Fluorescence microplate-readers and flow cytometry offer a wide variety of possibilities to measure apoptosis. Different methods have been established and implemented, some of which are based on the release of cytoplasmic components in the culture supernatant, the decrease in metabolism observed in dying cells, the uptake of vital dyes by dead cells, or on membrane-, mitochondrial-, or nuclear-changes occurring in apoptotic cells. Frequently used are DNA-binding dyes such as propidium iodide³⁵ or Hoechst dye, terminal deoxynucleotidyl transferase (TdT)-mediated end-labeling of the DNA strand breaks³⁴ detection of phosphatidyl serine on apoptotic cell membranes with Annexin V³⁶, DNA fragmentation laddering on agarose gels, detection of enzymological activities such as caspases, or simply visualization of apoptotic cells under the microscope.

Green fluorescence protein (GFP) from the jellyfish Aequorea victoria is widely used to monitor gene expression and protein localization in living organisms in vivo and in vitro²⁰⁻²³ GFP-fluorescence is stable, can be monitored noninvasively in living cells and persists in paraformaldehyde-fixed cells. FACS-optimized mutants of green fluorescence protein have been developed⁸. One of these mutants (GFPmut1) has been integrated into the pEGFP vectors and is commercially available (Clontech, Palo Alto, USA). The big advantage of this mutant is that the maximal excitation peak of GFPmut1 is 488 nm and the emission maxima is 507 nm. Conventional fluorescence microplate-readers and flow cytometers are appropriately equipped with light sources emitting light at 488 nm and have the suitable detection systems already installed, making the GFPmut1-protein an ideal candidate for microplate-reader based studies, flow cytometry and fluorescence microscopy. GFP has been used as a marker protein to detect cells transiently transfected with the commercially available plasmid pEGFP-Cl (Clontech)²⁴, According to this publication apoptosis was detected by reduced fluorescence of the DNA-binding dye PI in the apoptotic subpopulation. It was not recognized that GFP itself could be used as a marker for apoptosis.

A further well-known use of GFP is the FRET technology. FRET (fluorescence resonance energy transfer) can be detected by microscopy, flow cytometry and in fluorescence plate readers. Tandem molecules of green fluorescent proteins stably expressed within cells can serve as a genetically encoded sensor of protease activity. By using this technology it is possible to screen for agents, which modulate caspase activities²⁵.

Luo et al.²⁸ describe within the aforementioned FRET technology a method that is useful for correlating caspase-3 activation with apoptotic events and for rapid screening of potential drugs that may target the caspase dependent apoptotic process. However, Luo et al. were able to demonstrate said caspase-3 activation only upon UV-induced apoptosis in HeLa cells.

Also Tawa et al.²⁹ describe the use of caspase activation for a quantitative analysis of fluorescent GFP-based caspase substrate cleavage in cells by FRET and identification of novel inhibitors of apoptosis. The herein described technology is restricted and only applicable in the identification of caspase dependent apoptotic conditions, a differentiation between e.g. apoptotic and necrotic conditions is not possible.

The paper of Jones et al., supra (²⁵) describes a GFP-FRET intracellular caspase assay for drug screening. However, the same argument applies as for the Tawa technology: it only allows for a monitoring of caspase 3 dependent apoptosis.

The method described by Mahajan et al.³⁰ resembles that of Jones et al., however the Mahajan technology allows for an in vivo monitoring of apoptosis dependent on caspases 1 and 3.

Steff et al.²⁷ describe an assay for the detection of a decrease in green fluorescent protein for the monitoring of cell death. Said assay is supposedly amenable to high throughput screening technologies. However, with the here-described assay it is not possible to make a clear distinction between non-, pro-, or anti-apoptotic, or proliferative or necrotic activity of test compounds on test cells. However, such a distinction is essential for the evaluation of a compound eventually useful for diagnostic or therapeutic purposes. Furthermore, the method described by Steff et al. includes various cell handlings, such as a washing and resuspending step of the cells used in the assay. Said washing and resuspending procedure is carried out after induction of apoptosis has taken place. The cells then have to be transferred from the reaction vial into a 96-well plate. Such a handling of the cells unnecessarily elongates the whole procedure and may introduce artifacts which influence negatively the assay, e.g. loss of apoptotic cells during the washing step.

The present inventors were already able to show, that stable transfection of eukaryotic cells (e.g. A20.2J) with the pEGFP-C1 (as received from the manufacturer Clontech with the cytomegalovirus CMV promoter) results in little or no expression of the GFPmut1 gene (PCT/IB99/00030). Thus, a new vector for a stable transfection of test cells, which was able to induce a high expression of the GFP protein in selected cell lines was constructed by the combination of the known hEF-1α promoter, and the new combination of the CMV and the MOLV-LTR promoter (PCT/IB99/00030). Already then it was of particular interest to investigate the apoptotic or necrotic conditions of normal and cancer cells under the influence of test compounds and/or physical stimuli indicated by the amount of fluorescence emitted by cells transfected with the new vector. However, a fast and inexpensive high throughput screening method clearly differentiating between said non-, pro-, or anti-apoptotic or proliferative or necrotic effects of the compound to be tested in said cells, including primary cells, cell lines and spheroids has not been available yet.

### SUMMARY OF THE INVENTION

Thus, it is the major aspect of the present invention to provide an improved screening method, that allows for a high through put screening of compounds to be tested for their non-, pro-, or anti-apoptotic, or proliferative or necrotic effects on tumour cell lines or more particularly on primary cells, cell lines or spheroids of various origins representing healthy tissue- and/or organ-models or disease models. Said improved screening method has to be industrially applicable on a cost effective level.

More importantly, the main embodiment of the present invention allows for a clear and unambigous assessment of non-, pro-, or anti-apoptotic, or proliferative or necrotic activity of test compounds on test cells, which is achieved by a two-step screening assay:

A primary screening step is carried out in order to clearly discriminate between two main groups of different activities. The first group represents the pro-apoptotic and/or necrotic activity of test compounds, whereas the other group represents the non- and/or anti-apoptotic and/or proliferative activity of test compounds.

The secondary screening then allows the clear discrimination between pro-apoptotic and necrotic activity, and between non-, anti-apoptotic and proliferative activity.

Another major embodiment of the present invention a single cell imaging scanning system with an appropriate throughput capacity is applied as fluorescence detecting device in the primary screening, which enables a clear discrimination already at this level between non-, pro, or anti-apoptotic, or proliferative or necrotic activity of test compounds.

In yet a further embodiment the method of the present invention is applied for drug screening.

A further important aspect of the invention is the applicability of the herein described method for toxicological studies by assaying the necrotic/toxic activity of test compounds.

The screening method according to the present invention comprises stably transfecting a group of cells, either cells of a tumor cell line, or cells applicable as healthy tissue-and/or organ-model or disease model such as cell lines and primary cells of human or animal origin isolated from healthy individuals/animals or patients/animals suffering of diseases such as cancers, autoimmune diseases, organ transplantation derived pathogenesis, cardiovascular diseases and degenerative diseases of various origin or the like or spheroids with a vector coding for and expressing a marker protein in the respective cells or cell line. The transfected cells are transferred into 96 or 384 or 1536 well plates in a suitable culture medium. Afterwards the respective test compound is added in series of dilutions. The presence and/or activity, i.e. the decrease or increase, respectively, of the expressed marker protein in said group of cells is monitored by conventional methods, and compared with the results observed with a parallel group of the same test cells, which was not exposed to the test compound. With the screening method of the present invention an average of several hundreds to thousands test compounds can be evaluated per day.

These and other features, aspects, and advantages of the invention will become better understood with reference to the accompanying Figures, the description and appended claims.

### SHORT DESCRIPTION OF THE FIGURES

FIG. 1 shows the assessment of induced apoptosis and necrosis in A20GFP cells measured by a fluorescence microplate reader.

FIG. 2 shows the primary screen of 16 commercially available anti-cancer drugs on different EGFP expressing cell lines.

FIG. 3 demonstrates the discrimination between apoptotic and necrotic activity of anti-cancer drugs in a secondary screening on HeLa cells in relation to possible mechanisms of actions.

FIG. 4 demonstrates the correlation between relative fluorescence activity measured by FACScan versus fluorescence plate reader after induction of apoptosis.

FIG. 5 shows the anti-apoptotic activity of the pan caspase inhibitor zvADfmk after induction of apoptosis in JurkatGFP cells by serially diluted soluble FasL.

FIG. 6 demonstrates the reproducibility and profiling of several cell lines towards standard drugs in 96 well plates.

FIG. 7 shows the reproducibility and profiling of several cell lines towards standard drugs in 384 well plates.

### DETAILED DESCRIPTION OF THE INVENTION

The present invention relates to the assessment of the non-, pro-, or anti-apoptotic or proliferative or necrotic activity of test compounds on test cell systems of various origins in an industrially applicable assay for purposes such as drug screening and toxicology studies. Test compounds can have different activities on the test cells, i.e. non-, pro-, or anti-apoptotic, or proliferative and/or necrotic activity.

In a primary screening, the overall fluorescence activity of the test cells within a single well is measured with an appropriate fluorescence-detecting device, e.g. a fluorescence plate-reader. An appropriate cut-off value is set in order to clearly discriminate between two main groups of different activities. The first group represents the pro-apoptotic and/or necrotic activity of test compounds, whereas the other group represents the non- and/or anti-apoptotic and/or proliferative activity of test compounds.

In a secondary screening, the single-cell fluorescence activity of each test cell within a population of test cells is measured with an appropriate fluorescence detecting device, e.g. flow cytometry, microfluidic devices (chip technology) and single cell imaging scanning systems. This measurement allows the clear discrimination between pro-apoptotic and necrotic activity, and between non-, anti-apoptotic and proliferative activity.

The combination of primary and secondary screening allows the clear assessment of non-, pro-, or anti-apoptotic, or proliferative or necrotic activity of test compounds on test cells.

A single cell imaging, scanning systems with an appropriate throughput capacity can also be used as fluorescence detecting device in the primary screening, in order to clearly discriminate at that level between non-, pro, or anti-apoptotic, or proliferative or necrotic activity of test compounds.

Test compounds comprise synthetic or natural compounds, chemical or peptide structures or a combination thereof, proteins or recombinant proteins, pure compounds or a combination of pure compounds or extracts, such as plant extracts, extracts of marine micro- and macro-organisms and extracts of microbial fermentations. Test compounds are incubated with test cells either alone or in combination with known pro- or anti-apoptotic compounds or stimuli of various origins.

The test cell system comprises either a single cell, a single-cell population comprising cells of identical origin, a mixed-cell population comprising cells of different origin, or cells in spheroid form either of single-cell or mixed-cell population as defined above. Useful test cells are cell lines or primary cells from various origin and comprise eukaryotic and prokaryotic cells. Prokaryotic cells include bacterial and cyanobacterial cells. Eukaryotic cells include mammalian, fungal, insect, avian, worm, fish, crustacean, reptilian, amphibian and plant cells as well as cell lines thereof. Test cells usable in the method are cells of any type, preferentially normal, i.e. genetically non-altered, infected, e.g. with virus, parasites, bacteria, fungi or prions, tumor cells or genetically manipulated or altered cells of human, animal or plant origin, which can be cultured in vitro and/or in vivo. Human and animal primary cells as well as cell lines that have different origins and are derived from different tissues and/or organs such as liver, kidney, spleen, heart, lung, brain, blood, skin, muscles, bladder, myeloid and lymphoid system, reproductive system, visual system, bone marrow, gut, small intestine, mucosa, stomac, esophagous, duodenum, colon, pancreas, connective, embryonal and fetal tissue among others may be used. Useful test cells are cells applicable as healthy tissue- and/or organ-models or disease models, such as cell lines and primary cells of human or animal origin isolated from healthy individuals/animals or patients/animals suffering of diseases such as cancers, autoimmune diseases, organ transplantation derived pathogenesis, cardiovascular diseases and degenerative diseases of various origin, e.g. neurodegenerative diseases, or the like or spheroids.

Furthermore, appropriate test cells as described above are used for the purpose of toxicological studies, e.g. hepatotoxicological studies, kidney toxicity, skin toxicity, peripheral and central neurotoxicity, embryonal and fetal toxicity, toxicity of the spleen, heart, lung, blood, skin, muscles, bladder, myeloid and lymphoid system, reproductive system, visual system, bone marrow, gut, small intestine, mucosa, stomac, esophagous, duodenum, colon, pancreas, among others, where the necrotic/toxic activity of test compounds is assayed.

Microplate formats useful for the method are customized formats and standard formats such as 24-,48-, 96-, 384-, 1536- and any intermediate size well microplates, among others, as well as chip technology, where living cells can be used and a multitude of tests can be run at the same time.

Therapeutic fields of interest are cancer including angiogenesis, autoimmune and transplantation derived diseases, cardiovascular and degenerative diseases of various origin, such as neurodegenerative diseases, inflammation and allergic diseases, diseases of the reproductive system, dermatological applications and related diseases, among others.

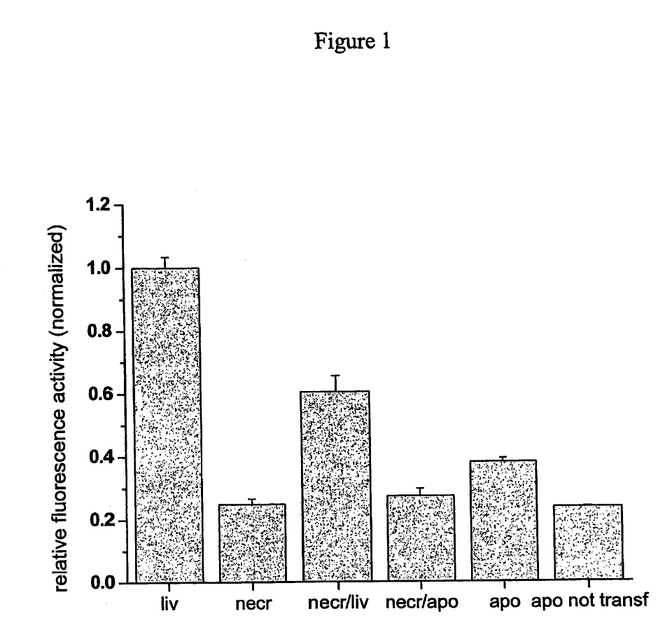

In FIG. 1, apoptosis and necrosis was induced in A20GFP cells and determined by using a fluorescence microplate reader, which measures the overall fluorescence activity within a single well. For this purpose A20GFP cells were either incubated with FasL for induction of apoptosis or with an anti-A20 antibody plus complement in order to induce necrosis. After incubation at standard conditions, the cells have been plated out into a 96-well plate. In the case where cell-mixtures, containing cells with different cell status, of either apoptotic and necrotic or living and necrotic cells were measured, equal amounts of apoptotic, necrotic or living-cells were pipetted appropriately into the same well (in a ratio of 1:1). Subsequently, the fluorescence activity was measured with a fluorescence microplate reader.

It could be observed that the homogeneous (unmixed) population of necrotic A20GFP cells lost almost entirely the fluorescence activity and showed values comparable to non-transfected control cells. Homogeneous (unmixed) living or apoptotic or necrotic cell populations can be clearly distinguished from each other due to a significant difference of the overall fluorescence activity of each cell-population, as shown in FIG. 1. On the other hand, cell-mixtures, i.e. subpopulations of apoptotic and necrotic or living and necrotic cells within the same well, show intermediate fluorescence activities when measured on a fluorescence microplate reader. This intermediate fluorescence activity varies depending on the contribution of each subpopulation, i.e. living, apoptotic and necrotic cells, to the overall fluorescence activity.

This experiment clearly demonstrates, that homogeneous (unmixed) populations of apoptotic or necrotic or living cells within a single well can be clearly distinguished from each other. On the contrary, subpopulations of apoptotic and/or necrotic and/or living cells within a single well, which globally show an intermediate fluorescence activity can hardly be distinguished from each other when measured on a fluorescence microplate reader.

Indeed, the use of cellular assays in drug screenings/high throughput screenings under real conditions shows that in most cases intermediate overall fluorescence activities are measured, which correspond to cell-mixtures within a single well. In conclusion, the need for a reliable and industrially applicable assay able to clearly discriminate between these different activities, i.e. non-, pro-, or anti-apoptotic, or proliferative or necrotic activity of test compounds on test cells, is evident.

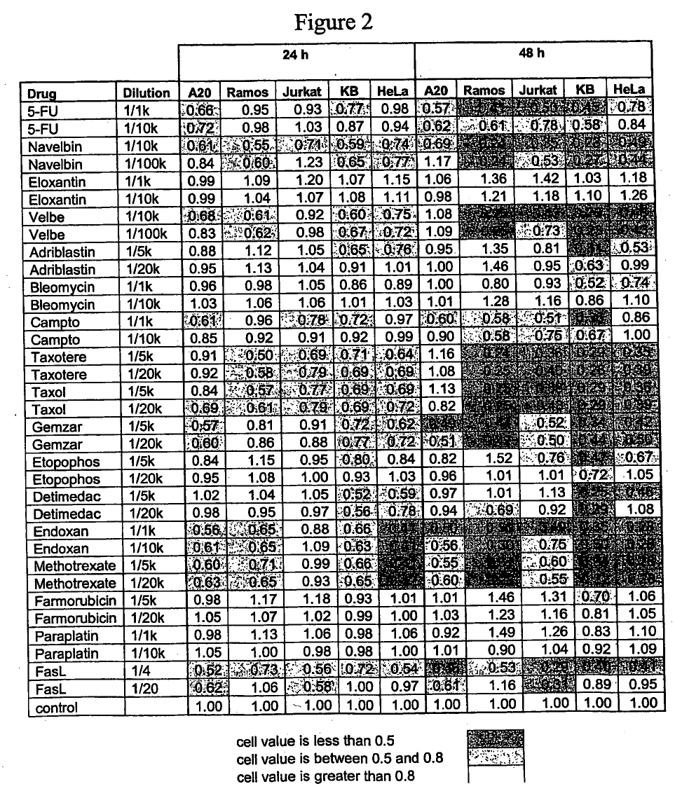

A representative primary screening with standard experimental set-up, where 16 commercially available anti-cancer drugs have been tested on five different EGFP expressing cell lines (HeLa, KB, A20, Ramos and Jurkat) in 96-well microtiter plates is shown in FIG. 2. Differential susceptibility regarding induction of apoptosis and/or necrosis after treatement with 5-Fluorouracil (5-FU), Gemzar, Methotrexate, Eloxantin, Detimedac, Endoxan, Paraplatin, Navelbine, Velbe, Taxotere, Taxol, Adriblastin, Bleomycin, Campto, Etopophos and Farmorubicin is shown. Measurements after 24 h and 48 h were performed with a fluorescence microplate reader, which measures the overall fluorescence activity in a single well. Used compound concentrations are indicated by a fraction of the original concentration.

FIG. 2 demonstrate that each tested cell line shows a specific pattern of sensitivity towards the tested drugs. A20 demonstrates the most refractory behaviour and KB the highest sensitivity towards the drug treatment. Beside a clear activity-dosage relation, a specific kinetic behaviour can be observed as well.

The setting of a cut-off value, corresponding to 0.8 in this case, allows the clear discrimination between two main groups of different activities. The first group with values under 0.8 represents the pro-apoptotic and/or necrotic activity of test compounds, whereas the other group with values above 0.8 represents the non- and/or anti-apoptotic and/or proliferative activity of test compounds. A representative example is Navelbine and Adriblastin, which show intermediate values of 0.49 and 0.53, respectively, when tested on HeLa cells at the highest concentration, i.e. 1/10 k for Navelbine and 1/5 k for Adriblastin. According to these intermediate values between 0 and 0.8 measured by a fluorescence microplate reader, it can hardly be discriminated between the subpopulations of apoptotic and necrotic cells within the same well.

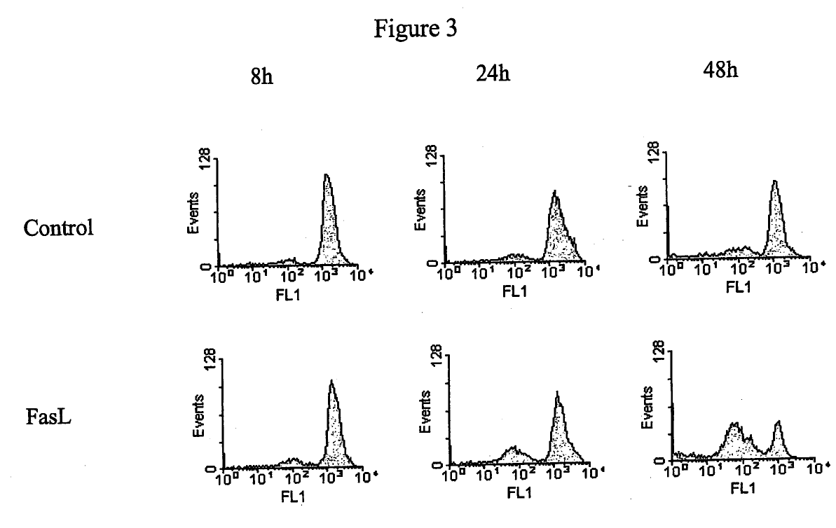

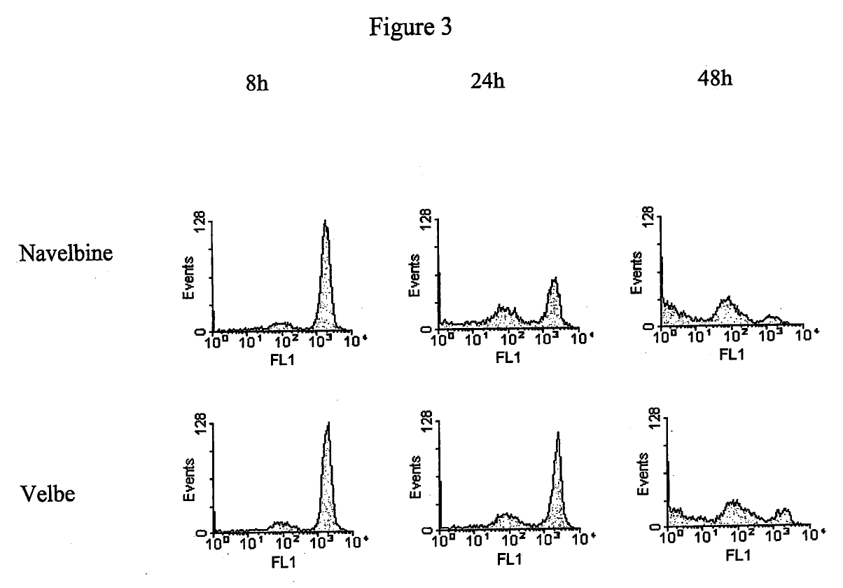

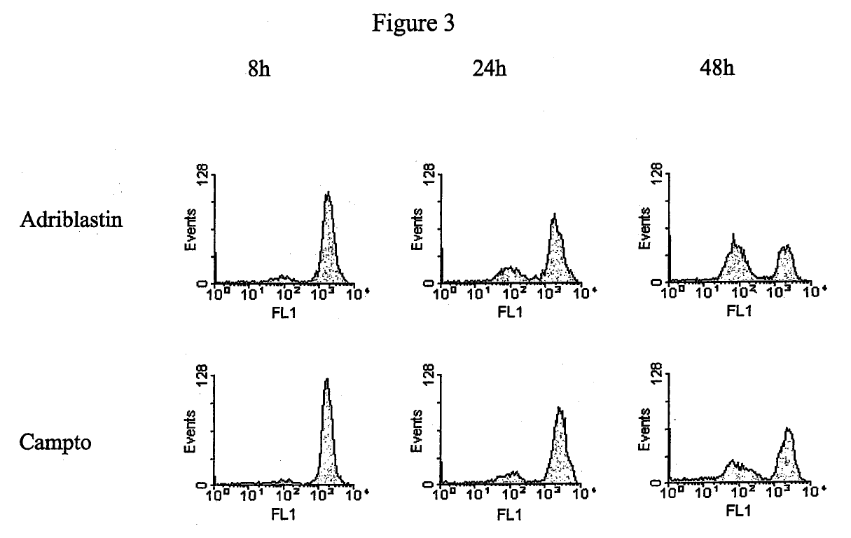

The unambiguous discrimination between the pro-apoptotic and necrotic activity of anti-cancer drugs on test cells within the same well, e.g. HeLa cells, in relation to possible mechanisms of actions is demonstrated in FIG. 3. For this purpose HeLa cells have been treated with typical anti-cancer drugs of different mode of actions. Navelbine and Velbe, two typical representatives of microtubules interfering drugs and Adriblastin and Campto, topo-isomerase inhibitors, which interfere with DNA synthesis.

FIG. 3 shows histograms of FACS analysis of cells treated either with Navelbine, Velbe, Adriblastine or Campto. Arrows indicate regions with either necrotic cells (left arrow) or apoptotic cells (middle arrow) or proliferating/living cells (right arrow). In this representative example, Navelbine and Adriblastin, which showed comparable overall fluorescence activities in the microplate reader as described above, demonstrate different activities when measured on a single-cell detecting device, such as flow cytometry. Indeed, Navelbine show a strong tendency to induce necrosis, whereas Adriblastin only induce apoptosis. Generally, the cell cycle inhibiting drugs, Navelbine and Velbe, respectively, show a strong tendency to induce necrosis in a certain fraction of cells. This fact became evident after exposure of the cells for longer than 24 h. In contrary, cells treated either with Campto or Adriblastin, typical DNA replication interfering compounds, do not show any tendency for necrotic activity even after exposure for 48 h. This experiment shows that the use of a single-cell fluorescence detecting device, e.g. flow cytometry, allows a clear discrimination between the subpopulation of apoptotic, necrotic and living/proliferating cells within the same well, after treatment of test cells with test compounds.

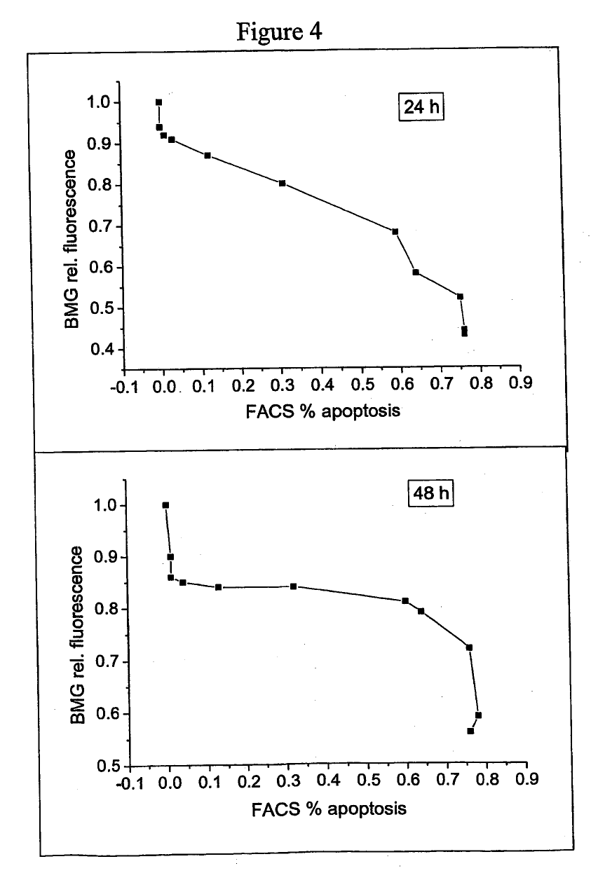

The differential sensitivity of the fluorescence microplate reader and flow cytometry, respectively, to changes in the fluorescence signal caused by apoptotic stimuli of various origin, is demonstrated in a representative experiment shown in FIG. 4. As already mentioned before, the fluorescence microplate reader measures global fluorescence activities in a single well in contrary to flow cytometry, e.g. a FACScan, which measures single-cell fluorescence activities. According to this different physical property, the sensitivity to changes in the fluorescence signal vary depending on the fluorescence detecting device used. In order to investigate the correlation between the apoptotic activity measured by FACScan (reflected directly by the population which shows reduced fluorescence) and the relative fluorescence activity measured by a fluorescence microplate reader, JurkatGFP cells have been induced to undergo apoptosis by treatment with serially diluted soluble FasL. The experiment has been carried out in 96-well microtiter plates and FACS tubes (BD Biosciences) in parallel. The samples have been measured after 24 h and 48 h, respectively, either in a BMG Fluostar fluorescence microplate reader (rel. fluorescence activity, vertical values) or with a FACScan (% apoptosis, horizontal values) and plotted versus each other. The correlation between apoptotic activities measured by FACScan and relative fluorescence activities measured by a fluorescence microplate reader turned out to change in a time dependent manner. This is illustrated in FIG. 4, which demonstrates the correlation at 24 h (FIG. 4A) and at 48 h (FIG. 4B), respectively. This time depedent change parallels the differential change in sensitivity towards apoptotic activities of the fluorescence microplate reader and the FACScan, respectively. A few percent of apoptotic cells (1-5%) measured by flow cytometry at 48 h, e.g. a FACScan, correlate to a reduction of approximately 20% in the relative fluorescence activity measured by the microplate reader. In other words, a change in the fluorescence signal of 20% measured by a microplate reader is hardly detectable by flow cytometry, e.g. a FACScan, which correspond to about 5% of apoptotic cells. A sharp change in the slope of the correlation curve is observed for the range of 5-65% apoptotic cells measured by flow cytometry, which correspond to a reduction in the relative fluorescence activity measured by a microplate reader of only 10%. This range of apoptotic activities of test compounds can be accurately measured by flow cytometry, e.g. a FACScan. Another drastic change in the slope of the correlation curve is observed in the range of 65-80% apoptotic activity measured by flow cytometry, which correspond to a further reduction of the relative fluorescence activity measured on the microplate reader of approximately 20%.

In conclusion, small amounts of apoptotic cells within the same well containing cell-mixtures of apoptotic and living/proliferating cells can be easily detected by the use of a fluorescence microplate reader. On the contrary, small amounts of apoptotic cells within the same well containing cell-mixtures are hardly detectable by flow cytometry, e.g. a FACScan. This behaviour has practical implications for the screening procedure. As already described, the primary screening step is performed with a fluorescence microplate reader. Consequently, this ensures that even small amounts of apoptotic stimuli of various origin can be detected by setting appropriate cut-off values and thereby acts as a highly reliable filter tool which detects even small apoptotic activities of various origin decreasing the likelyhood of missing active test compounds. Test compounds positively identified in the primary screening are then subjected to the secondary screening which is run on a flow cytometer. Flow cytometry is based on single-cell fluorescence measurements and demonstrates a high and reliable sensitivity in the range of 5-65% of apoptotic activity, in contrary to the microplate reader, and therefore enables the reliable quantification of apoptotic activity of test compounds.

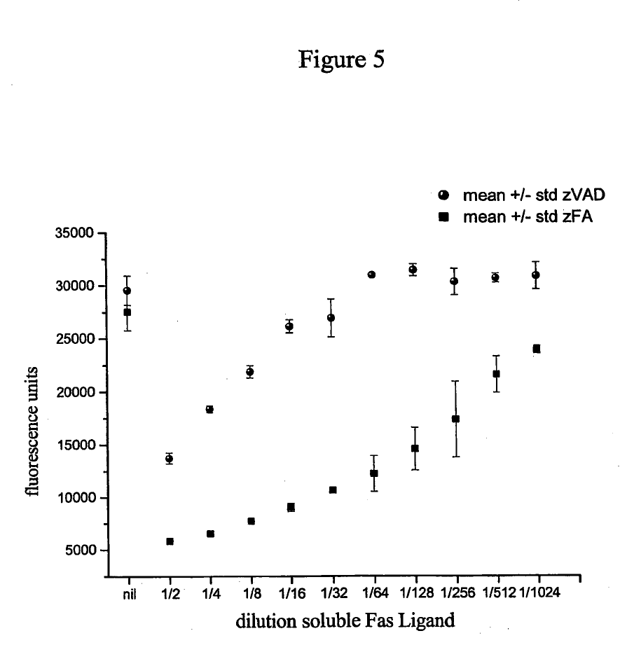

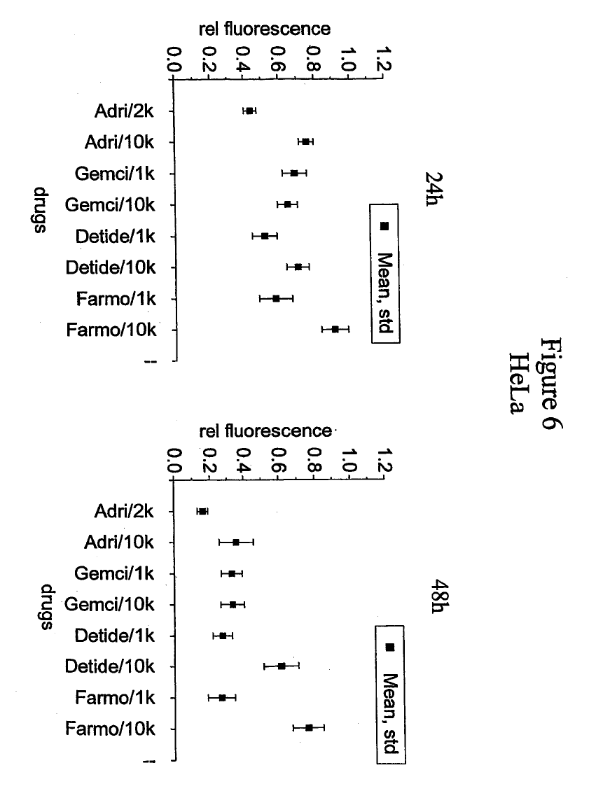

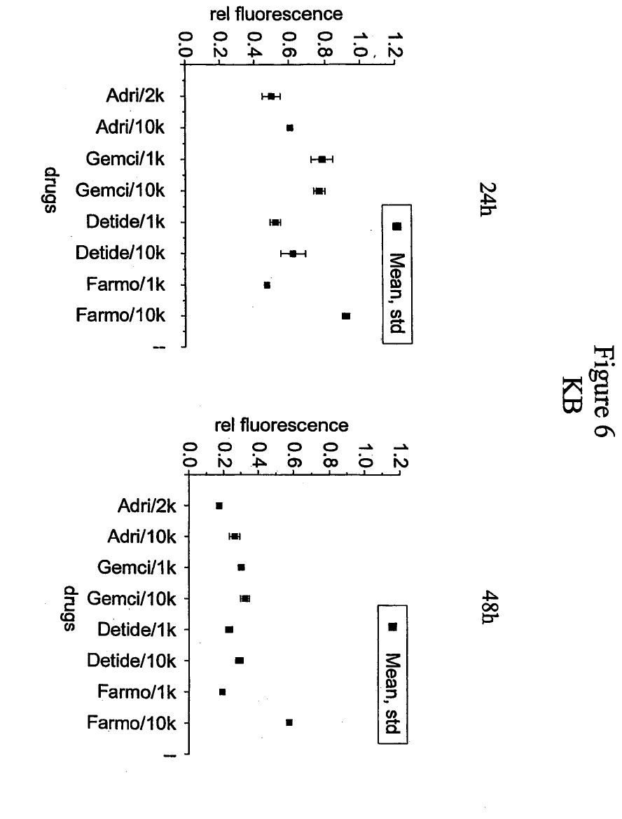

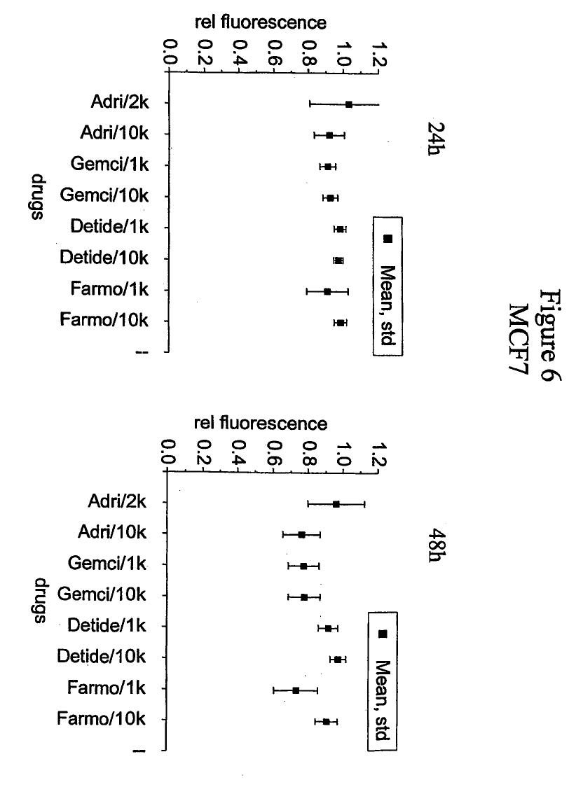

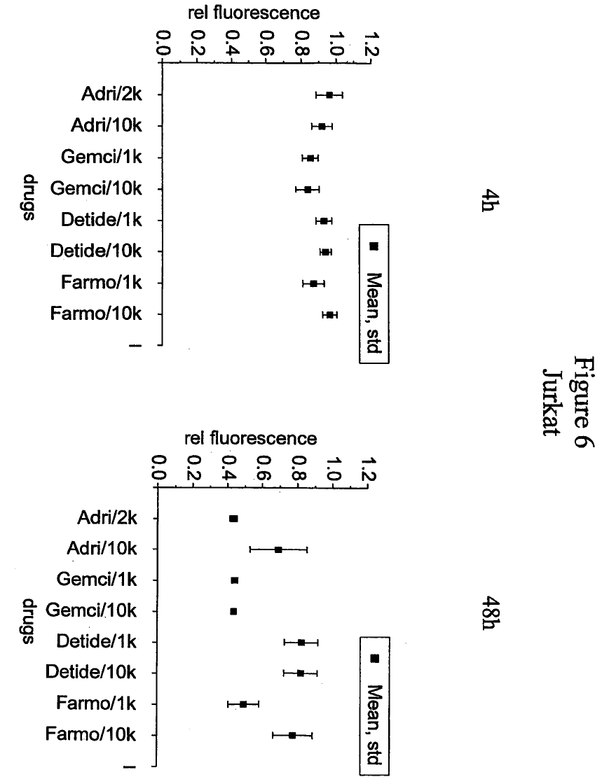

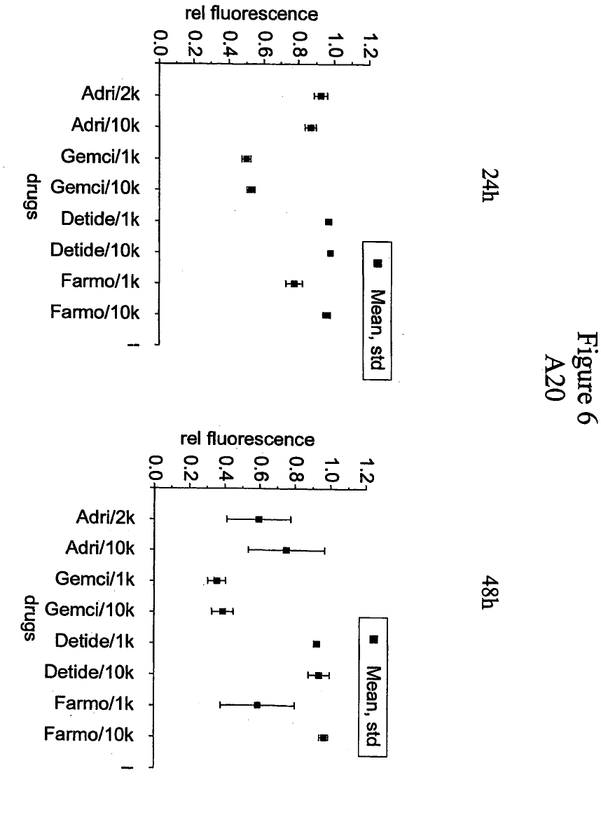

The activity of an anti-apoptotic compound, e.g. the pan caspase inhibitor zVADfmk, has been tested on JurkatGFP cells after induction of apoptosis by serially diluted soluble FasL and measured by a fluorescence microplate reader. A clear-cut dose-response behaviour, dependent on the dilution of FasL, can be observed already after 24 h of incubation at standard conditions. The pan caspase inhibitor zVAD, in contrary to its control peptide zFA, shows a clear inhibitory anti-apoptotic effect on the apoptotic activity of FasL. FIG. 5 shows representative data, which have also been observed with other EGFP transfected cells. The bars indicate standard deviations, which have been determined by measuring 6 single values for each datapoint.

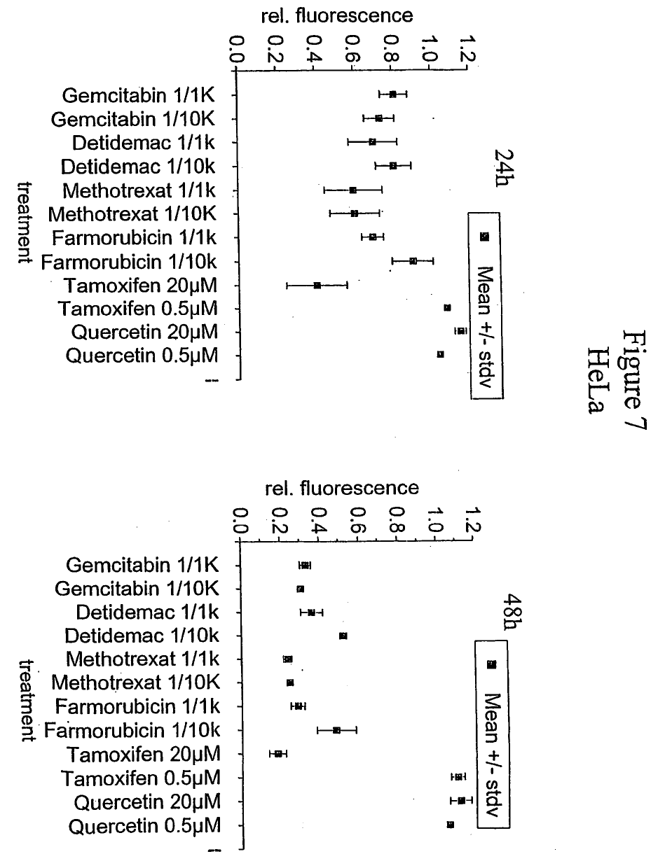

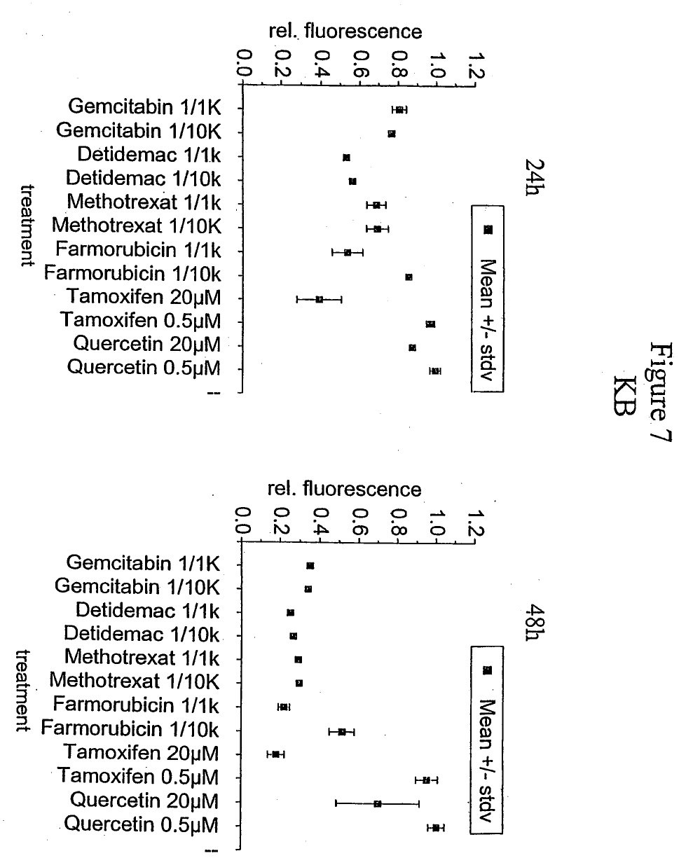

The reproducibility and the profiling of several cell lines towards standard drugs in 96 well plates is shown in FIG. 6.

Five different cell lines have been tested several times independently in a defined experimental setup, which correspond to the standard procedure for running the tests in 96-well plates. The following drugs were used: Adriblastin, Gemcitabin, Detimedac and Farmorubicin, respectively. Used cells are the following: HeLa, KB, MCF7, A20 and Jurkat.

Mean values and standard deviations are indicated for 24 h and 48 h measurements.

FIG. 6 shows that the system is robust and provides highly reproducible results. Therefore this experimental system is well suitable for high throughput screening in the 96-well format.

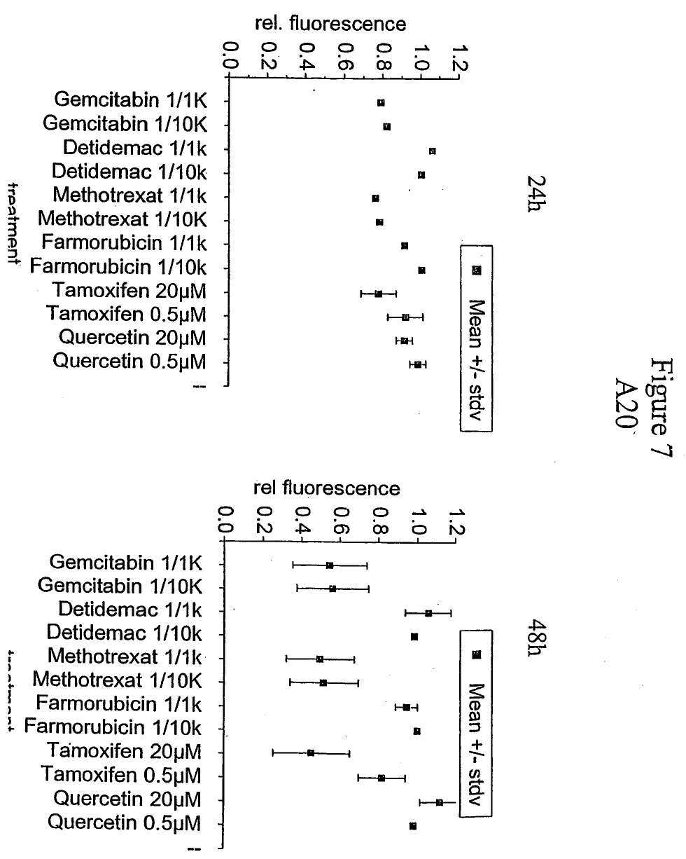

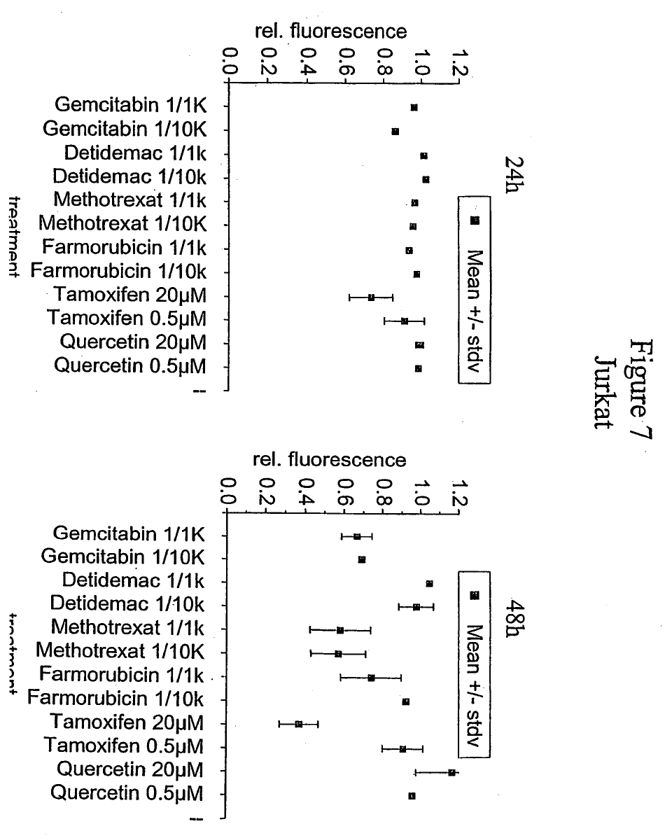

FIG. 7 demonstrates the reproducibility and profiling of several cell lines towards standard drugs in 384 well plates. Four different cell lines have been tested several times independently in a defined experimental setup which correspond to the standard procedure for running the tests in 384 well plates. The following drugs were used: Gemcitabin; Detimedac, Methotrexate and Farmorubicin, Tamoxifen and Quercetin. Used cells are the following: HeLa and KB; A20 and Jurkat.

Mean values and standard deviations are indicated for 24 h and 48 h measurements.

FIG. 7 show that the system is robust and provides highly reproducible results. Therefore, this experimental system is well suitable for high throughput screening in the 384 well format.

Beside the various above: mentioned cell lines, spheroids as three-dimensional cell models for screening non-, pro- or anti-apoptotic or proliferative or necrotic activity of test compounds may be used as well.

a) Pro-apoptotic activity of test compounds has been investigated in tumor models where cells are growing as spheroids that are nearly as heterogeneous as tumor nodules in vivo, regarding cell proliferation, resting cells, necrosis and apoptosis. There is also heterogeneity, where spatial distributions and therapeutical effects of anticancer drugs are concerned. The effects of different drugs vary dramatically between different types of spheroids and between different cell layers inside the spheroids. The influence of the oxygen pressure, pH and nutrition gradients on drug sensitivity of cells in spheroids is probably similar to the influence in tumor microregions. Therefore, spheroids will be of immense interest in the field of experimental tumor chemotherapy.

Instead of using EGFP expressing monolayer cell cultures for high throughput screening, spheroids are prepared either consisting of tumor cells only or mixed with untransfected stromal cells (e.g. fibroblast cell lines). As a result of the GFP reporter exclusively expressed in the tumor cells it is possible to monitor their behaviour in terms of necrosis, apoptosis and proliferation. This can be achieved by measuring the fluorescence activity in an appropriate device, e.g. in a fluorescence microplate reader or a single-cell imaging scanning system, as has been described for adherent and suspension cell cultures.

b) In order to investigate the anti-apoptotic activity of test compounds in diseases where the affected or target cells and organs are prone to undergo apoptosis (e.g. cardiovascular diseases like stroke or degenerative diseases found in neuronal- and muscle tissue pathogenesis), the appropriate cell models representing the disease in question can be used for preparing spheroids. By altering oxygen pressure, nutrient supply, pH or with the addition of certain active molecules (being responsible for degenerative processes) the EGFP transfected reporter and disease representing cells can be stimulated to undergo necrosis or apoptosis which can be easily monitored by its change in fluorescence activity. Compounds to be tested for anti-apoptotic or anti-necrotic activity can be added to the cells and the change in fluorescence activity can be tested.

### EXAMPLES

General Methods

### Example 1

Cell Cultures and Cell Lines.

The cell lines were cultured in RPMI-1640 tissue culture medium containing either 5% or 10% fetal calf serum, 0.05 mM 2-mercaptoethanol, 2 mM Glutamine and Penicillin/Streptomycin 50 g/ml (complete medium) (Sigma, Buchs, Switzerland). General growth conditions were 37° C. and 7.5% CO₂.

The following mouse cell lines were used: A20.2J (ATTC: TIB-208), PB3c (mastocyte cell line³²), MC57G (ATCC: CRL-2295):

The following human cell lines were used: HeLa (ATCC: CCL-2), KB (ATCC: CCL-17), MCF7 (ATCC: HTB-22), SK-BR-3 (ATCC: HTB-30), DM and HBL (melanoma cell lines³⁷), SK-Mel 1 (ATCC: HTB-67), SK-Mel 28 (ATCC: HTB-72), HaCaT (transformed keratinocytes³³), PC-3 (ATCC: CRL-1435), SW 480 (ATCC: CCL-228), NCI-H460 (ATCC: HTB-177), NCI-H1792 (ATTC: CRL-5895), HT1080 (ATTC: CCL-21), Jurkat (ATTC: TIB-152), Ramos (ATTC: CRL-1596), Raji (ATTC: CCL-86), H9 (ATTC: HTB-176), Hut78 (ATTC: TIB-161), K562 (ATTC: CCL 243) and HL-60 (ATTC: CCL 240)

Tumor cell spheroids have been prepared according to standard procedures³¹

### Example 2

Vector Construction and Transfection.

The vectors used in the present invention, which are either termed pEGFP-N1+MoLV-LTR and pBluescriptIIKS(+)+EF-1α+EGFP, respectively, have already been described elsewhere (PCT/IB99/00030). Any other commercially available and for the purposes of the present invention suitable vector may also be used. The vectors have been amplified and purified according to standard procedures and by the use of commercially available purification kits.

Transfection of the cells has been performed either by electroporation or with the help of liposomal reagents. Jurkat, A20.2J and the PB3c cell lines have been electroporated (PCT/IB99/00030) whereas all the other cell lines have been transfected by using Dotap transfection reagent (Roche Molecular Biochemicals, Switzerland) according to the manufacturer's protocol.

### Example 3

Selection, subcloning and analysis of the cells expressing EGFP.

The transfected cells were selected in 1 mg/ml G418 (Gibco BRL, Life Technologies AG, Basel, Switzerland) containing medium for 2-4 weeks. Selected suspension cells have then been sorted on a FACSVantage flow cytometer (BD BioSciences, Allschwil, Switzerland) for high EGFP expression profiles and subcloned by the limiting dilution method. Adherent cells positive for EGFP expression have been picked with pipette tips under sterile conditions and subsequently been subcloned twice by limiting dilution. In each case the resulting clones were expanded and subsequently characterized for homogeneous expression of EGFP in a FACScan (BD BioSciences) equipped with an argon laser tuned to 488 nm to excite EGFP, and a 515/545 bandpasss filter to monitor the green fluorescence emitted by the EGFP. Analysis was done by the CELLQuest program. In each measurement 10.000 events were collected.

### Example 4

Assay Set-Up.

All the manipulations were done under sterile conditions. The assays have been performed in commercially available 96 or 384 well flat bottom clear microtiter plates (Greiner, Germany) respectively, which are suitable for tissue culture techniques.

A defined number of adherent cells (96 well plates: 10⁴-10⁵, 384 well plates: 1500−2*10⁴) have been plated out 24 h before treatment either in 75 μl (96 well plates) or 60 μl (384 well plates) complete medium per well in order to ensure appropriate spreading before start of the treatment. For this purpose a peristaltic pump (e.g. Multidrop by Thermo-Labsystems, Finnland) or another suitable device was used. Cells in suspension have been plated out according to the same procedure but 1 h prior to treatment. Between seeding out and treatment or addition of compounds the cells were incubated at 37° C. under 7.5% CO₂. Subsequently, the compounds under investigation were added at defined concentrations dissolved either in 25 μl or 20 μl complete medium with an appropriate device (e.g. liquid handling system, multichannel pipette etc).

Immediately after the addition of the compounds to the cells the zero fluorescence value (t=0 h) was determined by using a fluorescence microplate reader in order to be able to normalize the fluorescence activities. Afterwards, the testplates were further incubated for a total of 48 h at 37° C. under 7.5% CO₂ and were shortly removed only for the purpose of measurement at 8 h, 24 h and 48 h, respectively.

### Example 5

Quantitation of EGFP Fluorescence Activity.

Relative fluorescence activities of EGFP in treated cells in relation to control cells were measured by using a BMG Fluostar microplate fluorescence reader equipped with a filterpair for excitation/emission at 485 nm/520 nm. The optimum signal to noise ratio was detected by using the time-resolved mode of measurement with a delay of 20 μs and an integration time over 1 ms. The gain was adjusted in such a way that the control cells produced a fluorescence activity of 90% of the maximum. Kinetics were performed by measuring the relative fluorescence activities at t=0 h, 8 h, 24 h and 48 h. Crude fluorescence activities were first individually normalized for different cell numbers seeded per well by dividing each value from t=8 h, 24 h and 48 h by the value of t=0 h. In a second step the resulting values, which were normalized for different cell numbers have been normalized in relation to the appropriate controls (cells only treated with dissolving agent, e.g. 1% DMSO, at the appropriate time) by forming the quotient. The normalization procedure, which accounts for variations of various origins (e.g. optical characteristics of individual plates, different growth properties due to serum variations, etc) allows to compare individually performed experiments among each other for statistical and other purposes.

### Example 6

Compounds, Chemical Agents and Treatments.

1) Four different classes of cytotoxic agents (a: antimetabolites, b: alkylating agents, c: cell-cycle inhibitor, d: DNA breaker (topo-isomerase inhibitor, intercalator, strand breaker), e: mixtures thereof, f: compounds interfering with the signal transduction pathway, such as caspase activity modifiers, agonists and antagonists of cell death receptors, modifiers of nucleases, phosphatases and kinases, which are commonly used in anticancer therapies have been extensively tested on several EGFP expressing cell lines for apoptotic/necrotic activities. They have been provided as stock solutions in ampoules.

Class a: 5-Fluorouracil, ICN, 50 mg/ml; Gemzar, Eli Lilly, 50 mg/ml; Methotrexate, Spitalapotheke Kantonsspital Basel, 4 mg/ml.

Class b: Eloxantin, Sanofi-Synthelabo, 5 mg/ml; Detimedac, Medac, 10 mg/ml; Endoxan, 1 mg/ml; Paraplatin, Bristol-Meyers Squibb, 10 mg/ml.

Class c: Navelbine, Robapharm, 10 mg/ml; Velbe, Eli Lily, 10 mg/ml; Taxotere, Aventis, 10 mg/ml; Taxol, 6 mg/ml.

Class d: Adriblastin, Pharmacia-Upjohn, 1 mg/ml; Bleomycin, Asta-Medica, 1 mg/ml; Campto, Aventis, 20 mg/ml; Etopophos, Bristol-Meyers Squibb; 5 mg/ml; Farmorubicin, Spitalapotheke Kantonsspital Basel, 2.5 mg/ml; Hycamtin, Smith-Kline Beecham, 1 mg/ml.

All these compounds have been used in clinical formulations as they were used for treating cancer patients. In the various tests they have been diluted with complete medium in the range of 1/1000 to 1/100000. They were kindly provided by the Department of Oncology, University Hospital of Basel, Switzerland.

2) Dexamethasone, Actinomycin D, Phorbol-Myristate-acetate, Cyclosporin A, Etoposide, Quercetin, Tamoxifen have been purchased by Alexis Corporation, Switzerland. Caspase inhibitor z-VAD-fmk and its control z-FA-fmk have been purchased by Enzyme Systems, Dublin, CA, USA.

3) Cells were also treated with soluble FasL and/or TRAIL, respectively, which is described elsewhere (PCT/IB99/00030).

Specific Methods

### Example 7

Assessment of Induced Apoptosis and Necrosis in A20GFP Cells Measured by a Fluorescence Microplate Reader.

A20GFP cells were either incubated with FasL for induction of apoptosis or with an anti-A20 antibody plus complement in order to induce necrosis. After 24 h incubation at standard conditions, 100 μl, which correspond to 200.000 cells have been plated out into a 96 well plate. In the case where mixed populations were measured, two times 50 μl were pipetted together into the same well. Subsequently, fluorescence activity was measured with a microplate reader.

### Example 8

Primary Screen of 16 Commercially Available Anti-Cancer Drugs on Different EGFP Expressing Cell Lines.

Five different cell lines (HeLa, KB, A20, Ramos and Jurkat) have been tested in 96 well microtiter plates for differential susceptibility regarding induction of apoptosis towards treatment with 5-Fluorouracil (5-FU), Gemzar, Methotrexate, Eloxantin, Detimedac, Endoxan, Paraplatin, Navelbine, Velbe, Taxotere, Taxol, Adriblastin, Bleomycin, Campto, Etopophos and Farmorubicin. Measurements after 24 h and 48 h were performed with a fluorescence plate reader. Used compound concentrations are indicated by a fraction (1/xk meaning 1/x000) of the original concentration.

Each tested cell line demonstrates a specific pattern of sensitivity towards the tested drugs. A20 demonstrates the most refractory behaviour and KB the highest sensitivity towards the drug treatment. Beside a clear activity-dosage relation a specific kinetic behaviour could be observed as well. The results are summarized in Table 1.

### Example 9

Secondary Screening: Discrimination Between Apoptotic and Necrotic Activity of Anti-Cancer Drugs in HeLa Cells in Relation to Possible Mechanisms of Actions.

HeLa cells have been treated with typical anti-cancer drugs of different mode of actions. Navelbine and Velbe, two typical representatives of microtubules interfering drugs and Adriblastin and Campto, topo-isomerase inhibitor, which interfere with DNA synthesis. Histograms of FACS analysis of cells treated either with Navelbine (1/1000 diluted), Velbe (1/1000 diluted), Adriblastin (1/2000 diluted) and Campto (1/1000 diluted) are shown in FIG. 4. Arrows indicate regions with either necrotic cells (left arrow), apoptotic cells (middle arrow) and proliferating cells (right arrow). The cell cycle inhibiting drugs, Navelbine and Velbe, respectively, show a strong tendency to induce necrosis in a certain fraction of cells. This fact became evident after exposure of the cells for longer than 24 h. In contrary, cells treated either with Campto or Adriblastine, typical DNA replication interfering compounds, do not show any tendency for necrotic activity even after exposure for 48 h.

### REFERENCES

1. Wyllie, A. H., et al. Int. Rev. Cytol. 68, 251 (1980)
2. Arends, M. J., et al. Int. Rev. Exp. Pathol. 32, 223 (1991)
3. Wyllie, A. H., Nature 284, 555 (1980)
4. Roy, C., et al. Exp. Cell Res. 200, 416 (1992)
5. Itoh, N., et al. Cell 66, 233-43 (1991)
6. Watanabe, F. R., et al. J. Immunol. 148, 1274-9 (1992)
7. Oehm, A., et al. J. Biol. Chem. 267, 10709-15 (1992)
8. Cormack, B. P., et al. Gene 173, 33-38 (1996)
9. Trauth, B. C., et al. Science 245, 301-5 (1989)
10. Itoh, N., et al. Cell 66, 233-43 (1991)
11. Watanabe, F. R., et al. J. Immunol. 148, 1274-9 (1992)
12. Ogasawara, J., et al. J. Exp. Med. 181, 485-91 (1995)
13. Suda, T., et al. S. J. Exp. Med. 179, 873-9 (1994).
14. Suda, T., et al. J. Immunol. 154, 3806-13 (1995)
15. Vignaux, F., et al. J. Exp. Med. 181, 781-6 (1995)
16. Tanaka, M., et al. Nature Med. 2, 317-322 (1996)
17. O'CONNELL, J., et al. J. Exp. Med. 184, 1075-1082
18. Hahne, M., et al. Science 274, 1363-1366 (1996)
19. Strand, S., et al. Nature Med. 2, 1361-1366 (1996)
20. Chalfie, M., et al. Science 263, 802-805 (1994)
21. Wang, S., et al. Nature 369, 400-403 (1994)
22. Prasher, D. C., et al. Gene 111, 229-233 (1992)
23. Inouye, S., et al. FEBS Letters 341, 277-280 (1994)
24. Lamm, G. M., et al. Nucleic Acids Research 25.(23), 4855-4857 (1997)

25. Jones, R., et, al. J. Biomol. Screen. 5 (5), 307-318 (2000)
26. Jeremias, I., et al. Eur. J. Immunol. 28, 143-152 (1998)
27. Steff, A. M., et al. Cytometry 45 (4), 237-243 (2001).
28. Luo, K. Q., et al. Biochem. Biophys. Res. Commun. 283 (5), 1054-1060 (2001)
29. Tawa, P., et al. Cell Death Differ. 8 (1), 30-37 (2001)
30. Mhajan, N. P., et al. Chem. Biol. 6 (6), 401 (1999)
31. Spheroid Culture in Cancer Research, CRC Press, Inc., 2000 Corporate Blvd., N.W., Boca Raton, Fla., 33431 (1992)
32. Ball, P E., et al. Differentiation 24, 74-78 (1983)
33. Boukamp, P A., et al. J Cell Biol Mar. 106 (3), 761-71 (1988)
34. Gavrieli, Y., et al. J. Cell Biol. 119 (3), 493-501 (1992)
35. Nicoletti, I., et al. J Immunol Methods 139 (2), 271-9 (1991)
36. Vermes, I. C., et al. J Immunol Methods 184 (1), 39-51 (1995)
Zhang, R. D., et al. Cancer Res 51 (8), 2029-2035 (1991)

1. A high throughput screening method for assaying non-, pro- or anti-apoptotic or proliferative or necrotic activity of test compounds in cells using vectors coding for a fluorescent marker protein and test cells transfected with said vector comprising a primary and a secondary screening step, wherein the primary screening step comprises measurement of the overall fluorescence activity of the test cells within a single well with a fluorescence detecting device, and wherein the secondary screening step comprises measuring of single-cell fluorescence activity of each test cell within a population of test cells with a fluorescence detection device.

2. (canceled)

3. The screening method of claim 1, wherein the marker protein is the GFP protein or GFPmut1 protein.

4. The screening method of claim 1, wherein the test cells are derived from healthy individuals/animals or patients/animals with diseases selected from the group consisting of degenerative diseases, cancer diseases, autoimmune and/or inflammatory diseases, cardiovascular diseases and neurological disorders.

5. The screening method according to claim 1, wherein the test cells are selected from the group consisting of Jurkat, HeLa, A20, KB, MCF7, Ramos, SK-MEL-1, SK-MEL-28,PC-3,NCI-H460,NCI-H1792, Raji, SK-BR-3, HaCaT, DM, HBL, SW480, HT-1080, HBL-100, Hs578T, MDA-MB-330, C-33A, BT-474, MDA-MB-133-VI, MDA-MB-157, MOLT-4, K-562, HCT-8, SW620, SW480, LoVo, SW403, SW1471, HL-60, HUT 78, H9, U937, Hep G2, PLC/PRF/5, Hs 683, U-138MG, A172 cells.

6. The screening method according to claim 1, wherein said test cells are spheroids.

7. (canceled)

8. (canceled)

9. The screening method according to claim 1, wherein the fluorescence detecting device in the primary screening step is a fluorescence plate reader.

10. (canceled)

11. The screening method according to claim 1, wherein the primary screening step discriminates on one side between pro-apoptotic and/or necrotic activity and on the other side non- and/or anti-apoptotic and/or proliferative activity of test compounds.

12. (canceled)

13. The screening method according to claim 1, wherein the fluorescence detecting device in the secondary screening step is selected from the group consisting of flow cytometry, microfluid (chip) devices, and single cell imaging scanning systems.

14. (canceled)

15. (canceled)

16. The screening method according to claim 1, wherein the compound to be tested is selected from the group consisting of synthetic or natural compounds, chemical or peptide structures or a combination thereof, proteins or recombinant proteins, pure compounds or a combination of pure compounds or plant extracts, extracts of marine micro- and macro-organisms and extracts of microbial fermentations.

17. The screening method according to claim 16, wherein the compound to be tested is a therapeutic/diagnostic agent selected from the groups comprising:
a) antimetabolites;
b) alkylating agents;
c) cell-cycle inhibitors;
d) DNA breaker;
e) mixtures thereof;
f) compounds interfering with the signal transduction pathway.

18. The screening method according to claim 1, wherein the test cell system comprises cell lines or primary cells in form of single cells, single-cell populations of same origin, mixed-cell populations of different origin or spheroid cell forms.

19. The screening method according to claim 18, wherein the test cell system comprises eukaryotic cells selected from the group consisting of mammalian, fungal, insect, avian, worm, fish, crustacean, reptilian, amphibian and plant cells.

20. The screening method according to claim 19, wherein said eukaryotic cells are genetically non-altered cells, cells infected with virus, parasites, bacteria, fungi or prions, tumor cells or genetically manipulated or altered cells of human, animal or plant origin.

21. The screening method according to claim 19, wherein said eukaryotic cells are derived from human or animal tissues and/or organs selected from the group comprising liver, kidney, spleen, heart, lung, brain, blood, skin, muscles, bladder, myeloid and lymphoid system, reproductive system, bone marrow, gut, small intestine, mucosa, stomac, esophagous, duodenum, colon, pancreas, connective, embryonal and fetal tissue.

22. (canceled)

23. The screening method according to claim 18, wherein the test cell system comprises prokaryotic cells selected from the group consisting of bacterial and cyanobacterial cells.

24. (canceled)

25. Use of the screening method according to claim 1 for drug screening.

26. The use of the screening method according to claim 25, wherein the drug screening is applied in the therapeutic and diagnostic fields selected from the group comprising cancer including angiogenesis, autoimmune and transplantation derived diseases, cardiovascular and degenerative diseases of various origin, neurodegenerative diseases, inflammation and allergic diseases, diseases of the reproductive system, dermatological applications and related diseases.

27. The use of the screening method according to claim 25 for toxicological studies.

28. The use of the screening method according to claim 27, comprising assaying of necrotic activity of toxic compounds.

29. The use of the screening method according to claim 28, the toxicological studies being selected from the group comprising hepatotoxicological, kidney toxicity, skin toxicity, neurotoxicity, connective, embryonal and fetal toxicity studies, toxicity of the spleen, heart, lung, blood, skin, muscles, bladder, myeloid and lymphoid system, reproductive system, visual system, bone marrow, gut, small intestine, mucosa, stomac, esophagous, duodenum, colon and pancreas.
